(19) United States
(12) Patent Application Publication
(10) Pub. No.: US 2009/0104086 A1
(43) Pub. Date: Apr. 23, 2009

(54) Photocatalytic titanium dioxide nanocrystals

(76) Inventors: Adam Zax, Capistrano Beach, CA (US); Yasser Elassal, San Clemente, CA (US)

Correspondence Address: Vic Lin;Myers Dawes Andras & Sherman LLP, Suite 1150, 19900 MacArthur Blvd., Irvine, CA, 92612, US

(21) Appl. No.: 12/288,273

(22) Filed: Oct. 17, 2008

Related U.S. Application Data
(60) Provisional application No. 60/999,567, filed on Oct. 19, 2007.

(51) Int. Cl. A62B 11/00; B05D 1/36

(52) U.S. Cl. 422/121; 427/419.2; 422/122

(57) ABSTRACT

A photocatalytically active titanium dioxide film may be applied onto surfaces of a variety of objects to oxidize matter that comes into contact with those surfaces. Various methods may be used to apply a solution of the photocatalytically active nanoparticles onto surfaces receiving regular human contact or proximate to human presence. An inorganic primer layer may be initially applied to an organic substrate, such as food, plants, flowers and foliage, to prevent the photocatalytically active coating from oxidizing the organic substrate.

### CROSS REFERENCE TO RELATED APPLICATIONS

This application relates to and claims priority from U.S. Provisional Application Ser. No. 60/999,567 filed on Oct. 19, 2007 entitled “METHODS OF APPLICATION AND NOVEL USES FOR PEROXOTITANIUM ACID SOLUTION OF PHOTOCATALYTIC TITANIUM DIOXIDE NANOCRYSTALS,” the disclosure of which is incorporated herein by reference as if fully set forth.

### FIELD OF THE INVENTION

The present invention relates to photocatalytic compositions to oxidize matter.

### BACKGROUND OF THE INVENTION

Odors are caused by vapors and small particulates that float in the air and can be detectable by the human nose in a few parts per million. Indoor air also carries airborne pathogens and fungal spores that can cause disease when breathed. Volatile organic compounds and other chemical vapors can accumulate indoors and can adversely affect health.

Fungal spores can come to rest on exposed food surfaces and begin growing mold, contributing to spoilage. Bacteria also causes food spoilage in a process that consumes nutrients while producing waste that can sometimes be harmful (e.g. food poisoning). Another cause of food spoilage is ethylene, a gas produced by overripe fruit, which in turn accelerates the over-ripening of adjacent fruit.

Pathogens that are not airborne are still susceptible to transfer by contact. An infected person can inadvertently spread disease by contaminating doorknobs, countertops, and other shared surfaces with bacteria and viruses.

The common thread in all those problems is that they can be caused by tiny amounts of microscopic substances.

### SUMMARY OF THE INVENTION

In one aspect, a method is provided for applying a photocatalytically active titanium dioxide film. The method comprises applying an inorganic primer layer on an organic substrate, preventing oxidation of the substrate with the inorganic primer layer, and applying a solution of anatase titanium dioxide nanoparticles and an inorganic binder over the primer. The inorganic primer layer may be non-toxic and applied on a piece of food. The step of applying the solution of anatase titanium dioxide nanoparticles and the inorganic binder over the primer comprises applying a non-toxic solution of anatase titanium dioxide nanoparticles and a non-toxic inorganic binder over the primer. The inorganic primer layer may also be applied on plants, foliage, flowers or fruits.

The method further comprises catalyzing a production of carbon dioxide and water in order to promote growth. The film may be inactivated by blocking the film from light exposure. The method further comprises preventing spoilage, wilting, senescence, abscission and over-ripening of the plants, foliage, flowers or fruits. The method further comprises preventing fungal and bacterial growth on the surface of the plants, foliage, flowers or fruits.

In another aspect, a method for applying a photocatalytically active titanium dioxide film comprises identifying a surface receptive of human contact or adjacent to human presence. A solution of anatase titanium dioxide nanoparticles and an inorganic binder are applied to the surface. The method also comprises oxidizing matter on or adjacent to the surface. The method further comprises deodorizing and purifying air adjacent to the surface.

The method further comprises providing mold remediation on the surface, and preventing mold and mold spore growth on the surface. The method further comprises providing bacterial and viral remediation on the surface, and preventing a spread of disease. The surface may also be provided with self-cleaning and maintenance-reducing-properties. The solution of anatase titanium dioxide nanoparticles and an inorganic binder may be applied to the substrate during the manufacturing process. The solution of anatase titanium dioxide nanoparticles and the inorganic binder may be applied to a surface in situ.

In a further aspect, a method is provided for treating air in at least a partially enclosed space, such as packaging. The method comprises applying an inorganic binder to an object, applying a solution of anatase titanium dioxide nanoparticles to the object, and placing the treated object inside a package in order to treat air adjacent to the object. The method further comprises closing the package to enclose the air therein completely, and scavenging oxygen in the enclosed air by converting oxygen to carbon dioxide. The method further comprises deodorizing the air in the at least partially enclosed space.

In a further aspect, an oxidizing apparatus for use in enclosed spaces, such as packaging, is provided. The apparatus comprises an object, and a photocatalytically active titanium dioxide film applied to the object. The object can be added to a container in order to treat air adjacent to the object. The object may comprise a light emitting source, and may be powered by batteries or any other power source. The object may receive light from an outside source. The object may also absorb and later emit light. The object may also produce light as a result of a chemical reaction. The object may also produce light from radioactive decay.

Where the packaging is at least partially enclosed, the film may deodorize air in the at least partially enclosed package. Where the packaging is completely enclosed, the film may scavenge oxygen in the completely enclosed package. The object may be stackable.

Accordingly, a photocatalytically active titanium dioxide film may be applied onto surfaces of a variety of objects to oxidize matter that comes into contact with those surfaces. Various methods may be used to apply a solution of the photocatalytically active nanoparticles onto surfaces receiving regular human contact or proximate to human presence. An inorganic primer layer may be initially applied to an organic substrate, such as food, plants, flowers and foliage, to prevent the photocatalytically active coating from oxidizing the organic substrate.

### DESCRIPTION OF THE DRAWINGS

These and other features, aspects and advantages of the present invention will be more fully understood when considered with respect to the following specification, appended claims and accompanying drawings, wherein:

FIG. 1 is a photograph of lemon halves, one treated with a Preferred Solution and the other untreated.

### DETAILED DESCRIPTION OF THE PREFERRED EMBODIMENTS

Titanium dioxide nanoparticles act as photo catalysts that facilitate the oxidation of organic and inorganic matter adsorbed onto their surface in the presence of light. These nanoparticles can be incorporated into a solution with a binder that allows them to adhere to various surfaces in order to act as a photocatalytically active surface coating in applications where it is desirable to oxidize matter in contact with the surfaces.

Several variables affect the efficiency, practicality, safety, and environmental impact of such an application, including nanoparticle concentration, nanoparticle dispersion, nanoparticle surface area, nanoparticle crystalline structure, curing temperature, binder toxicity, binder corrosiveness, delivery method, component costs, and the maximum wavelength (lowest energy) of light that can activate the coating.

One method of forming a titanium dioxide film on a surface is to use a slurry of powdered titanium dioxide in a binder to coat and then bake onto a substrate. However, there have been many problems associated with using a titanium powder coating process. The baking temperatures required are typically quite high which limits the possible substrates to those that are resistant to heat. It can be extremely difficult or impossible to use that method in the field to create a titanium dioxide film on existing surfaces. The coatings used for this process generally comprise volatile organic compounds and acids to facilitate the dispersion of titanium dioxide particles. Again, this limits the possible substrates to those which are resistant to the particular binders used and can result in hazardous compounds being generated during the baking process. The effectiveness of the coating is highly dependent on the titanium dioxide nanoparticles used. Coatings made from titanium dioxide particles that are amorphous or crystallized in any form other than anatase will be ineffective as photo catalysts. Titanium dioxide particles are only photocatalytically active if they are very near the surface of the coating, so coatings that have poor titanium dioxide surface area exposure will be ineffective regardless of the percent mass titanium dioxide that they contain.

According to a preferred embodiment of the invention, a well-dispersed titanium dioxide anatase nanocrystal solution is produced from a peroxotitanium-based titanium dioxide-forming solution and mixed with a binder for providing a host of healthy and/or desirable properties such as: deodorizing properties; air purifying properties; mold remediation and the prevention of mold and mold spore growth; bacterial and viral remediation and prevention of the spread of organic based disease; field-applied, self-cleaning/maintenance-reducing properties; field-applied growth enhancement in plants and foliage; prevention of growth of mold and other microbes on plants, foliage, and fruit with a non-toxic barrier; a protective layer on plants and foliage as a means of preventing spoilage, wilting, senescence, abscission (leaves and flowers dropping off) and over-ripening; and an oxidizing protective layer on or as an attachment to transparent and translucent storage bags, linings and wraps, both bio-degradable and non-bio degradable, and containers used in the packing, handling and storage of plants, foliage, and fruit as a means of preventing spoilage, wilting, senescence, abscission (leaves and flowers dropping off) and over-ripening or spoilage.

A well-dispersed titanium dioxide anatase nanocrystal solution produced from a peroxotitanium-based titanium dioxide-forming solution and mixed with a binder (the “Preferred Solution” or “film” or “coating”) can be used to apply a film of titanium dioxide nanocrystals at room temperature, which makes it suitable for novel applications where baking is impractical. As examples and not by way of limitation, binders may comprise peroxotitanium and colloidal silica. One delivery method possible for that Preferred Solution is the use of a portable sprayer which makes field application very practical. The Preferred Solution is water-based so it is nonflammable. The Preferred Solution contains no volatile organic compounds and therefore will not release volatile organic compounds into the air when applied. The binder is not corrosive so it can be used to apply titanium dioxide films to substrates that are sensitive to strong acids. The binder allows for an excellent dispersion of titanium dioxide nanoparticles without the use of organic dispersants or strong acids. The titanium dioxide nanoparticles dispersed in the binder preferably comprise anatase nanocrystals that are photocatalytically active.

The Preferred Solution is compatible with several different delivery methods, allowing it to be customized to each novel application. The amount of Preferred Solution necessary per measure of area has been calculated for many different applications. The concentration and ratio of titanium dioxide nanocrystals and binder can be adjusted based on the desired application to efficiently deliver a titanium dioxide crystal film to a myriad of surfaces.

In the preferred embodiment, the Preferred Solution can be used to apply a thin titanium dioxide crystal film with a high ratio of surface area to mass, rendering the coating highly photocatalytic without discoloring the substrate and can even be used on transparent surfaces like glass or plastic. The Preferred Solution can be used safely on organic surfaces by first using the binder without titanium dioxide nanocrystals as a primer, then depositing the titanium dioxide crystal film on top of that, preventing the photocatalyst from oxidizing the organic substrate.

Due to the quality of the titanium dioxide nanocrystals and their dispersion in the binder, the titanium dioxide crystal films produced by the Preferred Solution are photocatalytically active to light with a wavelength preferably up to 490 nanometers, which is within the spectrum of visible light. While most titanium dioxide delivery methods produce films that are only active in the UV spectrum, the solution allows for novel applications of titanium dioxide crystal films that can be photocatalytically activated by a simple light source, such as indoor light bulb.

Previous attempts to overcome the aforementioned problems with titanium dioxide film coating make use of expensive or unstable materials. The Preferred Solution, however, uses highly stable materials to apply an efficient titanium dioxide crystal film, making it very cost effective in comparison.

In the preferred embodiment, a well-dispersed titanium dioxide anatase nanocrystal solution is produced from a peroxotitanium-based titanium dioxide-forming solution and mixed with a binder for novel applications of a photocatalytically active titanium dioxide crystal film that have become viable due to key advantages it has over previously known titanium dioxide formulations and delivery systems.

Disclosed herein are preferred methods for providing deodorizing properties; providing air purifying properties; providing mold remediation and the prevention of mold and mold spore growth; providing bacterial and viral remediation and prevention of the spread of organic based disease; providing field-applied, self-cleaning/maintenance-reducing properties; creating field-applied growth enhancement in plants and foliage; treating plants, foliage, and fruit with a non-toxic barrier that will prevent the growth of mold and other microbes; creating a protective layer on plants and foliage as a means of preventing spoilage, wilting, senescence, abscission (leaves and flowers dropping off) and over-ripening; and creating an oxidizing protective layer on or as an attachment to transparent and translucent storage bags, linings and wraps, both bio-degradable and non-bio degradable, and containers used in the packing, handling and storage of plants, foliage, and fruit as a means of preventing spoilage, wilting, senescence, abscission (leaves and flowers dropping off) and over-ripening or spoilage.

In one preferred method for applying a photocatalytically active titanium dioxide film, an initial step is identifying a surface receptive of human contact or adjacent to human presence that would benefit from a photocatalytic coating. In the preferred embodiment, these surfaces include, without limitation, automobiles; trucks; commercial vehicles; recreational vehicles; airplanes; subways; homes; commercial buildings; industrial buildings; restaurants; hospitals; amusement parks; doctors' offices; surgical centers; child day care centers; sports facilities; gymnasiums; amusement games including video games, pinball games, simulator games, mechanical, digital and computerized skill games; all surfaces found in any public or private restrooms or bathrooms including waterless and traditional urinals, toilets, bidets, sinks, faucets, countertops, walls and ceilings. Specific applications include walls, ceilings, picture frames and glass, ceiling fans, light fixture covers that are transparent and/or opaque, and shutters made of any type of material.

Treating ceilings with the titanium-dioxide nanocrystal solution has the advantage over many other locations in a room of rarely having any human or mechanical contact, and thus rarely has abrasion that can, over time, diminish the amount of photocatalytically active titanium dioxide nanocrystals that are on a treated surface. Interiors ceilings are also typically the brightest area of a room, providing more energy to the oxidation properties of the coating, increasing the coating's efficiency.

The Preferred Solution may be used for treating interior and exterior ceiling fans in all construction as a means of deodorizing the air by removing volatile organic compounds in the rooms and areas for which they are located. Like ceilings, ceiling fans offer an advantage over many other locations in a room in that it is an area that rarely has any human or mechanical contact and thus rarely has abrasion that can, over time, diminish the amount of photocatalytically active titanium dioxide nanocrystals on a treated surface. Ceiling fans create an automated means of circulating the air, forcing it to come into contact with the photocatalytically active titanium dioxide nanocrystals on a treated surface, such as the ceiling it is attached to or adjacent walls or furniture for example, as well as the blades and components of the fan itself. Ceiling fans are also typically located in the brightest area of a room with many models including light sources of their own; this provides more energy to the oxidation properties of the coating, increasing the coatings efficiency. Fans that circulate the air while exposed to light offer an excellent way to purify air in an area. By treating the rotating blades and the surrounding components, organic particles are drawn to the surface and oxidized by the coating.

The Preferred Solution may be used for treating bug screens and all types of fenestration, including doors, windows and skylights. Various screens made from aluminum, steel, vinyl, plastics or other polymers may be treated with the photocatalytically active Preferred Solution as a means of creating a natural filtration system for the home, vehicle or building in which it is installed. When polluted air is drawn through these screens by means of natural circulation or by manual/automated fans or vacuums, in the presence of light, the air is deodorized through the oxidation of the organic materials in the air that can cause odor.

The Preferred Solution may be used to treat all video games (e.g., consoles, controllers, and other equipment involving human contact), pinball games, simulator games, mechanical, digital and computerized skill games, all games that involve wired or wireless controllers that are not disposable after each use. All games, sports and hobbies that use balls and/or equipment that may be shared, including pool cues, swimming pool gear, waterslides and other water park amusements would benefit from being treated. All of these applications and locations are key areas where human contact spreads disease. While treating the components of these at the manufacturing level will solve some of the issues, there are now and will always be more existing surfaces to be treated than new ones produced each year. This makes the uniqueness of being able to treat these surfaces where they are in use, without the typically required heat sources, a major advantage over previous methods that could not enable it at all.

The preferred embodiment includes treating medical devices, hypodermic needles and all surgical instruments and tools used for medical purposes with the Preferred Solution. Bacterial and viral remediation and preventing the spread of disease caused by viruses, bacteria and other organisms is one of the most important jobs of any type of medical facility, from the hospital and surgery center to the average doctor's office. One of the most critical areas in terms of risk are those items, materials and tools that come in direct contact with the doctors, patients and healthcare workers. Some of these items include, but are not limited to: stethoscopes; hypodermic needles and syringes; scalpels; clamps and all other surgical tools; Intravenous (I.V.) drip machines; all machines used in diagnosis, such as MRI, CAT Scan and X-Ray devices; machines used in monitoring of patients, from heart monitors to breathing assist machines to blood pressure monitoring devices for example; and all machines used to regulate and cleanse, such as iron lungs, dialysis machines; wheelchairs, gurneys, and other modes of patient transport. With the growing number of cases of hospital-acquired MRSA, an anti-microbial coating of the Preferred Solution on all surfaces that come into human contact would save many lives.

The preferred embodiment also includes applying the photocatalytically active coating on non-medical objects located in hospitals which may be subject to frequent human contact, such as computer keyboards, telephones, door handles, writing utensils and even clipboards and other stationary products. Applying the Preferred Solution to the inner surface of windows in hospital rooms may also effectively kill germs, particularly since such windows would receive natural light for an extended period of time on a daily basis.

The preferred methods call for treating utensils for serving in public private and commercial applications including utensils shared at “buffet” style restaurants. Millions of people share serving utensils daily at restaurants and other food service establishments. This is unfortunately a very effective way of passing germs and viruses between humans and can be prevented by applying the Preferred Solution to these utensils at the manufacturing stage as well as treating existing utensils with field service “In-Situ” application. While treating the components of these during the manufacturing process will solve some of the issues, there are now and will always be more existing surfaces and utensils to be treated than new ones produced each year. Again, the uniqueness of being able to treat these surfaces where they are in use, without the typically required heat sources, provides a major advantage over previous methods that could not enable it at all.

In construction of all types of buildings, industrial, residential and commercial, all surfaces where common human to human contact is made may be treated, including doorknobs, handles, hand rails and guard rails where people support themselves or others using these items to gain or protect balance or achieve entry or exit, escalators and moving walkways and their hand rails. The preferred embodiment calls for treating escalators and moving walkways and their hand rails with the photocatalytically active titanium dioxide nanocrystal solutions. In many high population public locations such as airports and train stations, moving walkways are used to keep people traffic moving and free from bottlenecks. These escalators and moving walkways offer a major challenge in preventing the spread of communicable diseases, colds and viruses. Treating these areas will prevent the spread of those diseases by oxidizing the microscopic organisms prior to their being picked up by another person.

The preferred embodiment includes treatment of all safety masks and dust filters that are used to protect the health of mammals. These protective devices in many cases are the last line of defense in preventing the ingestion, inhalation or any other type of absorption of harmful irritants, pollutants, allergens and other volatile organic compounds.

The Preferred Solution can be used to creating field applied, self-cleaning/maintenance-reducing properties on glass, metals, polymers, plastics, woods, natural stones, marble, granite, quartz, stucco, concretes, cements, inorganic and organic paints. The preferred embodiment includes treating interior and exterior signs created from any type of material. Signs meant to convey messages become blurry, damaged and can be difficult to read, requiring premature replacement. Treating these signs prevents the organic materials in the atmosphere and surrounding pollutants from deteriorating the signs appearance as typically occurs over time on untreated signs. Treating thumbprint, fingerprint and hand print scanner lenses/print reading devices will prevent the “lifting”, counterfeiting or unauthorized use of the “print” left by the last user of each device.

The Preferred Solution can be used for treating the inside of greenhouses, sunrooms, aviaries and conservatories that contain plants and foliage. The treated walls, ceilings, roofs and planting containers/equipment use the light that enters these typically bright rooms to catalyze reactions that give off both water and carbon dioxide by products. While the amounts of carbon dioxide are too minimal to be considered an environmental problem, it is sufficient to support a noticeable increase in the growth rate and vitality of plants and foliage growing in or near these areas. The increase in growth and vitality, in turn, leads to the increase in oxygen output.

In another embodiment of the invention, a solution of inorganic nanoparticles like peroxotitanium or colloidal silica is used as a primer layer on organic substrates in order to be protected from the photocatalytically active titanium dioxide film applied afterwards. Many organic substrates can benefit from a photocatalytic coating that oxidizes foreign organic matter.

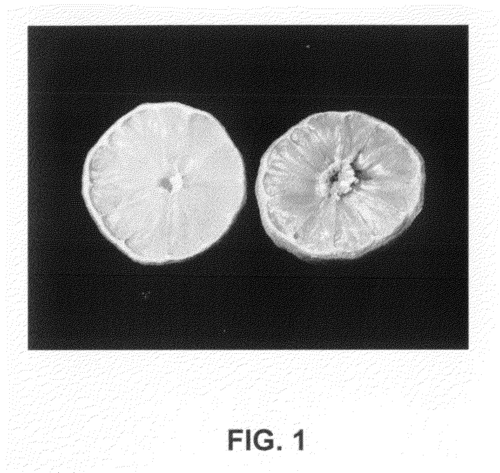

FIG. 1 illustrates two halves of the same lemon shown three weeks after being cut open. The one on the left has been treated with the Preferred Solution, while the one on the right has not been treated. An inorganic primer layer was first applied to the cut surface of the left lemon half prior to prevent the subsequently applied photocatalytic coating from oxidizing the lemon itself. In the case of food such as this, the coating is particularly useful as it is found to prevent mold and bacterial growth when exposed to light, but is safe for consumption because the nanoparticles become inactive once ingested due to the lack of light. In FIG. 1, the left lemon half coated with titanium dioxide nanoparticles was left on a counter next to window. Several weeks later, the treated half remained visibly mold-free while the untreated side was discolored with mold and dried out. This effect can be replicated inside a drawer or pantry with the help of a small light source, such as a UV bulb, in the frequency range that isn't harmful to humans.

In the case of plants, flowers and foliage, the coating can prevent harmful fungal growths and infection by plant pathogenic bacteria that are unable to survive the oxidation at the molecular level. Organic growths can destroy plants or foliage and/or attract insects and other unwanted animals that can also be detrimental to the health of the plant. Billions of dollars of plants and foliage are damaged or destroyed by organic growths that grow on them and deprive the plant of necessary nutrients, sun and/or by consuming the plant itself. The preferred embodiment includes using the Preferred Solution as a coating for plants and foliage which will prevent these growths from damaging the plants/foliage by oxidizing them before they can begin damaging them.

The coating also slows down senescence and abscission in plants by reducing the levels of ethylene gas at the surface. Flowers can maintain a healthy appearance longer if sprayed with the nanoparticles. The coating can encourage plant growth in some cases by acting as a carbon dioxide source.

In another embodiment, the coating can take advantage of partially enclosed or fully enclosed spaces. Accordingly, a preferred method for treating air in at least a partially enclosed space is provided. In partially enclosed spaces, the coating can act as an oxidizer. In fully enclosed spaces, the coating can act as an oxygen scavenger. In a vacuum-sealed package, there is likely still a small amount of air that can degrade oxygen-sensitive substrates. However, if the package is lined with the coating, it will catalyze the conversion of oxygen to carbon dioxide. If the packaged item is fruit, this serves the added benefit of breaking down ethylene, a gas that causes over-ripening and spoilage of fruit. The saying “one bad apple spoils the bunch” is a reference to the fact that a spoiling apple releases lots of ethylene, which in turn spoils nearby fruit. A photocatalytic coating will help prevent that. Specially coated translucent containers can be sold for use on countertops or in drawers or cabinets with built-in UV light sources.

However, the coating need not be on the packaging itself. A small apparatus, such as a disk or rod, can be coated with titanium dioxide and placed in the package with the fruit in order to break down ethylene and scavenge oxygen. If the apparatus is in the shape of a thin stackable object, like a disc, it could be packaged conveniently for sale, allowing a consumer to take advantage of photocatalysis in any translucent container simply by placing a small object inside. Other stackable objects include, for example, thin discs that are too large to fit in a child's mouth but small enough to be convenient can be stacked in a small box that easily fits inside a drawer, so that any translucent container can be imparted the effects of photocatalysis in the few seconds it takes to grab a disc and toss it inside.

If the container is not transparent, the photocatalytic effects of the coating can still be activated by including a light source in the package. If the light source itself is coated, it becomes a convenient way to take advantage of photocatalysis in any container with a single apparatus. While the oxygen scavenging effects are significant in a relatively small, enclosed space, the air-purifying properties of a small, coated object is applicable even to partially enclosed spaces, such as in a shoe, handbag, closet drawers, open containers, etc.

If a partially or fully enclosed space is not exposed to natural light, a variety of light emitting devices may be employed. The coating may be applied on the light emitting devices or on objects exposed to the light from the devices. For example, a coated UV bulb placed in a shoe at night can significantly reduce the odor levels inside. Similar results are achievable in bags, drawers, cabinets, and even closets. A simple light source, such as a UV bulb with a battery, provides a portable way to purify the air in an enclosed or partially enclosed space. Batteries may be used to power such light emitting devices.

Light sources may also comprise objects which produce light as a result of a chemical reaction. For example, the reaction of hydrogen peroxide with a phenyl oxalate ester will release energy that can cause a dye to emit light. If the dye chosen is 9,10-diphenylanthracene, it will emit blue light that be used by the photocatalyst. And if the light source is chemical in nature, like a glow stick, they can be sold as portable, disposable air purifiers. Long-lasting light sources can be created from radioactive materials as in betavoltaics or by encapsulating radioactive materials in protective shielding that photoluminesces in the UVA range or with wavelengths up to 490 nm.

Light sources may also comprise objects which absorb and then subsequently emit light. Phosphors can absorb light energy and continue to emit light even after the original source is removed. Zinc sulfide activated with silver is a phosphor that can be used to provide blue light for the photocatalyst. Europium-activated strontium haloborates are phosphors that can provide light in the UV range and can be used in narrow-band UV lamps for powering the coating in settings where it is undesirable to have visible light, such as a romantic restaurant.

In the preferred embodiment, the Preferred Solution may be applied onto surfaces in a variety of ways. For example, the Preferred Solution can be applied to a surface using a high volume, low pressure spray gun that atomizes the solution to provide a thin, consistent coating with excellent nanocrystal dispersion that remains transparent for uses including glass and clear plastics. This method is applicable in the field wherever it is convenient to have a source of compressed air or alternate non-hazardous gas.

If there is no compressed air source available but small droplet sizes are still required for clarity of the titanium dioxide crystal film, the Preferred Solution can be applied from a pressurized can, such as in an aerosol. If droplet size is less important, the Preferred Solution can be applied using a trigger sprayer or a pump sprayer, or even simply wiped onto the substrate using a microfiber cloth or a prepackaged moistened towelette.

An alternate method that provides both excellent atomization and dispersion involves a system that releases a Preferred Solution mist, applying a thin titanium dioxide crystal film to every exposed surface in an enclosed or partially enclosed space. This can be accomplished by using a heatless humidifier that atomizes the droplets mechanically.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

What is claimed is:

1. A method for applying a photocatalytically active titanium dioxide film, comprising:
applying an inorganic primer layer on an organic substrate;
preventing oxidation of the substrate with the inorganic primer layer; and
applying a solution of anatase titanium dioxide nanoparticles and an inorganic binder over the primer.

2. The method of claim 1, wherein:
the step of applying the inorganic primer layer on the organic substrate comprises applying a non-toxic inorganic primer layer on a piece of food; and
the step of applying the solution of anatase titanium dioxide nanoparticles and the inorganic binder over the primer comprises applying a non-toxic solution of anatase titanium dioxide nanoparticles and a non-toxic inorganic binder over the primer.

3. The method of claim 1, wherein:
the step of applying the inorganic primer layer on the organic substrate comprises applying the inorganic primer layer on plants, foliage, flowers or fruits.

4. The method of claim 3, further comprising:
catalyzing a production of carbon dioxide and water in order to promote growth.

5. The method of claim 3, further comprising:
inactivating the film by blocking the film from light exposure.

6. The method of claim 3, further comprising:
preventing spoilage, wilting, senescence, abscission and over-ripening of the plants, foliage, flowers or fruits.

7. The method of claim 3, further comprising:
preventing fungal and bacterial growth on the surface of the plants, foliage, flowers or fruits.

8. A method for applying a photocatalytically active titanium dioxide film, comprising:
identifying a surface receptive of human contact or adjacent to human presence;
applying a solution of anatase titanium dioxide nanoparticles and an inorganic binder to the surface; and
oxidizing matter on or adjacent to the surface.

9. The method of claim 8, further comprising:
deodorizing air adjacent to the surface.

10. The method of claim 8, further comprising:
purifying air adjacent to the surface.

11. The method of claim 8, further comprising:
providing mold remediation on the surface; and
preventing mold and mold spore growth on the surface.

12. The method of claim 8, further comprising:
providing bacterial and viral remediation on the surface; and
preventing a spread of disease.

13. The method of claim 8, further comprising:
providing the surface with self-cleaning and maintenance-reducing-properties.

14. The method of claim 8, wherein:
the step of applying the solution of anatase titanium dioxide nanoparticles and the inorganic binder to the surface comprises applying the solution of anatase titanium dioxide nanoparticles and the inorganic binder to the substrate during the manufacturing process.

15. The method of claim 8, wherein:
the step of applying the solution of anatase titanium dioxide nanoparticles and the inorganic binder to the surface comprises applying the solution of anatase titanium dioxide nanoparticles and the inorganic binder to the surface in situ.

16. A method for treating air in at least a partially enclosed space, comprising:
applying an inorganic binder to an object;
applying a solution of anatase titanium dioxide nanoparticles to the object; and
placing the treated object inside the space in order to treat air adjacent to the object.

17. The method of claim 16, further comprising:
closing the space to enclose the air therein; and
scavenging oxygen in the enclosed air by converting oxygen to carbon dioxide.

18. The method of claim 16, further comprising:
deodorizing the air in the at least partially enclosed space.

19. An oxidizing apparatus for use in an enclosed space, comprising:
an object;
a photocatalytically active titanium dioxide film applied to the object; and
wherein said object can be added to a container in order to treat air adjacent to the object.

20. The apparatus of claim 19, wherein the object comprises a light emitting source.

21. The apparatus of claim 19, wherein the object receives light from an outside source.

22. The apparatus of claim 19, wherein the space is at least partially enclosed, and wherein the film deodorizes air in the at least partially enclosed package.

23. The apparatus of claim 19, wherein the space is completely enclosed, and wherein the film scavenges oxygen in the completely enclosed space.

24. The apparatus of claim 19, wherein the object comprises a light-emitting source powered by batteries.

25. The apparatus of claim 19, wherein the object produces light from a chemical reaction.

26. The apparatus of claim 19, wherein the object absorbs and later emits light.

27. The apparatus of claim 19, wherein the object produces light from radioactive decay.

28. The apparatus of claim 19, wherein the object comprises a stackable object.
